(19) United States
(12) Patent Application Publication
(10) Pub. No.: US 2018/0192906 A1
(43) Pub. Date: Jul. 12, 2018

(54) POLYMER COMPOSITION AND ELECTRODE FOR A DEVICE FOR THE NON-INVASIVE MEASUREMENT OF BIOLOGICAL ELECTRICAL SIGNALS

(71) Applicant: RYTHM, Paris (FR)

(72) Inventors: Quentin SOULET DE BRUGIERE, Pyla sur Mer (FR); Hugo MERCIER, Chilly Mazarin (FR)

(21) Appl. No.: 15/742,078

(22) PCT Filed: Jul. 7, 2016
(86) PCT No.: PCT/EP2016/066131

§ 371 Date: Jan. 5, 2018

(30) Foreign Application Priority Data
Jul. 7, 2015 (FR) 15 56449

(51) Int. Cl. A61B 5/0478; A61B 5/00; C08K 7/24; C08K 3/04; A61L 31/12; A61L 31/04

(52) U.S. Cl. CPC A61B 5/0478; A61B 5/6803; C08K 7/24; C08K 3/041; C08K 3/042; C08K 3/04; C08K 2201/006; A61L 31/126; A61L 31/041; A61B 2562/0209; C08K 2201/001; C08K 2201/011; C08K 3/046

(57) ABSTRACT

The invention relates to polymer compositions including a polymer matrix in which are dispersed carbon nanotubes and adsorbent elements selected from activated carbon particles and graphene nanoplatelets, as well as electrodes including such compositions and electrical circuits and devices including the electrodes.

### FIELD OF THE INVENTION

The present invention relates to electrodes for devices for the non-invasive measurement of biological electrical signals.

More particularly, the invention relates to an electrode for a device for the non-invasive measurement of biological electrical signals which can be used without conducting gel, and a polymer composition which can be used in such an electrode.

### BACKGROUND OF THE INVENTION

An electrode for the measurement of electrical biological signals is used to detect and measure bio-signals which are for example representative of nerve activity such as brain activity or muscle activity.

Such biological electrical signals are used for example to obtain an electroencephalogram (EEG), electromyogram (EMG), electrooculogram (EOG), or electrocardiogram (ECG), this list not being exhaustive and having applications not only in the medical field but also in more mainstream uses such as recreation, personal monitoring, or exercise.

“Wet” metal electrodes are known, for example Ag/AgCl electrodes. These present numerous disadvantages including the requirement of skin preparation prior to contact involving hair removal and scraping the stratum corneum, an irritation to the skin during extended use in people with sensitive skin, or the presence of a gel between the skin and the electrode which is not very compatible with regular recreational use.

“Dry” metal electrodes are also known, for example from document U.S. Pat. No. 4,865,039. However, such electrodes have a moderate signal quality, require an expensive amplifier, and are uncomfortable during extended use.

Finally, dry polymer electrodes are known, for example from document U.S. Pat. No. 8,608,984, comprising a polyether-based thermoplastic polyurethane polymer in which are dispersed a styrene polymer and a conductive filler comprising a mixture of carbon nanotubes and carbon black.

The present invention is intended to improve the chemical properties of the interface and the mechanical and electrical characteristics of such electrodes and polymer compositions. The present invention also aims to provide a polymer composition and an electrode requiring no prior preparation of the skin such as washing or the presence of a gel in order to collect signals. Said electrode material can be used with or without direct contact with the skin. Furthermore, the present invention aims to provide a polymer composition and an electrode which can be easily adapted to the desired use by adjusting the mechanical, electrical, and chemical characteristics.

### OBJECTS AND SUMMARY OF THE INVENTION

The first object of the invention thus relates to a polymer composition comprising a polymer matrix in which are dispersed carbon nanotubes and adsorbents selected from activated carbon particles and graphene nanoplatelets.

In one embodiment, the polymer composition comprises 0.5 to 10 percent carbon nanotubes by weight, preferably 2 to percent carbon nanotubes by weight, based on the total weight of the polymer composition.

In one embodiment, the polymer composition comprises 0.5 to 30 percent adsorbents by weight, preferably from 2 to 15 percent adsorbents by weight, based on the total weight of the polymer composition.

In one embodiment, the activated carbon particles have a specific surface area greater than 300 m²/g, preferably greater than 500 m²/g, more preferably greater than 700 m²/g, even more preferably greater than 1000 m²/g.

In one embodiment, the polymer composition has a hardness within a range of 10 Shore A to 80 Shore A.

The invention also relates to an electrode for the non-invasive measurement of biological electrical signals, the electrode comprising a polymer composition as described above that is able to come into contact with living tissue.

In one embodiment, the polymer composition forms a first layer extending between a first face, able to come into contact with living tissue, and a second face opposite to the first face in a thickness direction, and the electrode further comprises a second layer of a conductive polymer arranged on the second face of the polymer composition.

In one embodiment, a thickness of the polymer composition, measured in the thickness direction, is smaller than a thickness of the conductive polymer, preferably at least two times smaller than a thickness of the conductive polymer.

In one embodiment, the conductive polymer has a hardness greater than a hardness of the polymer composition.

Another object of the invention relates to an electrical circuit for the non-invasive measurement of biological electrical signals, comprising:

an electrode as described above, comprising at least one electrical conductor connected to the polymer composition or to the conductive polymer, and

a unit for processing the signals measured by the electrode, connected to the electrical conductor of the electrode.

The invention also relates to a device for measuring brain waves suitable for wearing by a person, the device comprising a support member, adapted to at least partially surround the head of a person so as to be held thereon, on which is mounted at least one electrode as described above such that the polymer composition is able to come into contact with the skin of said person.

In one embodiment, the device comprises an electrical circuit as described above wherein the electrode and the processing unit are mounted on the support member.

Finally, the invention relates to the use of adsorbents selected from activated carbon particles and graphene nanoplatelets, in order to increase the ionic adsorption of a polymer composition comprising a polymer matrix in which carbon nanotubes are dispersed, said adsorbents being dispersed in the polymer matrix.

### BRIEF DESCRIPTION OF THE DRAWINGS

Other features and advantages of the invention will be apparent from the following description of several of its embodiments, given by way of non-limiting examples, with reference to the accompanying drawings. In the drawings:

FIG. 1 is a schematic view of an electrode according to a first embodiment of the invention,

FIG. 2 is a schematic view of an electrode according to a second embodiment of the invention, and

FIG. 3 is a schematic view of a device for measuring brain waves suitable for wearing by a person, according to an embodiment of the invention.

In the various figures, the same references designate identical or similar elements.

### MORE DETAILED DESCRIPTION

The polymer composition 1 according to the invention comprises a dispersion phase and a dispersed phase. The dispersion phase comprises a polymer matrix 2 and ensures the mechanical structure of the composition, while the dispersed phase comprises carbon nanotubes 3 and adsorbents 4 selected from activated carbon particles and graphene nanoplatelets and ensures the electrical conduction in the polymer composition and the electrochemical interface with the living tissue 9 in contact with the polymer composition 1 or the electrode 6.

The polymer matrix 2 may be a single polymer or a mixture of polymers. The polymer matrix is usually composed of one or a combination of polymers, silicones, and hydrogels. The polymer matrix is selected so as to have at least one or more of the following characteristics: flexible (hardness less than 65 Shore A), hydrophilic, permeable to Cl−, K+, and/or Na+ ions, compatible with the skin and non-allergenic, resistant to bacteria over a period of at least 6 months, washable with water and/or alcohol, and the addition of carbon has little impact on its mechanical properties. The polymer matrix is for example chosen from polymers having low glass transition temperatures.

The polymer matrix is, for example, chosen from a list of polymers such as: polyethylene glycol and particularly poly(oxyethylene) (PEG, PEO), polypropylene glycol and particularly polypropylene oxide (PPG, PPO), a copolymer of polyethylene glycol and polypropylene glycol, in particular a three-block copolymer such as a poloxamer, a polyvinyl alcohol (PVOH, PVA, PVA1), a silicone such as polydimethylsiloxane (PDMS), a derivative of cellulose and in particular of hydroxypropyl methylcellulose, hydroxypropyl cellulose, or methylcellulose (HPMC, HPC), a hydrogel.

Furthermore, the polymer matrix may contain polymer modifiers and other additives such as surfactants.

The polymer matrix 2 thus forms a flexible and amorphous material, for example an elastomeric polymer matrix.

The polymer matrix is preferably hydrophilic.

The polymer composition 1 further comprises carbon nanotubes 3 and adsorbents 4 selected from activated carbon particles and graphene nanoplatelets dispersed in the polymer matrix.

The carbon nanotubes 3 used in preparing the polymer compositions of the invention have for example a diameter within the range of 5 to 20 nanometers and a length within the range of 1 to 5 microns.

The carbon nanotubes used in preparing the polymer compositions of the invention have for example an aspect ratio within the range of 80 to 200 but can be up to 1000 or more.

The carbon nanotubes used in preparing the polymer compositions of the invention may be multiwall carbon nanotubes (MWNT) or single wall carbon nanotubes (SWNT).

The polymer composition of the invention further comprises adsorbents 4 selected from activated carbon particles and graphene nanoplatelets.

In one embodiment of the invention, the adsorbents 4 are activated carbon particles more specifically. In this manner, a particularly high specific surface area is obtained.

“Activated carbon” is understood to mean a material having undergone specific preparation in order to impart strong adsorbing capacity, in particular due to a very large specific surface area.

“Graphene nanoplatelets”, also known for example by the names graphene nanoflakes, graphene nanopowder, nanometric graphene platelets, or nanographene platelets, is understood to mean nanoparticles formed of graphene and consisting of small stacks of one to several layers of graphene, for example from 1 to 15 nanometers thick, with a diameter typically ranging from a few hundred nanometers to several hundred micrometers.

Graphene has a very high theoretical specific surface area (2630 m²/g), and graphene nanoplatelets therefore have very high specific surface areas as a result, and in particular specific surface areas close to those of activated carbon particles.

In the case of the present invention, the adsorbents used may be prepared so as to have strong adsorption of Cl−, K+, and/or Na+ ions at least.

The activated carbon particles may be prepared by carbonization (pyrolysis) and/or by activation/oxidation (exposure to an oxidizing atmosphere). The activated carbon particles may also, or before carbonization, be prepared by chemical activation by being impregnated with acid, strong base, or a salt.

The graphene nanoplatelets can be prepared in various ways known from the literature, for example in the manner indicated in the article “Processing of nanographene platelets (NGPS) and NGP nanocomposites: a review” by B. Z. Jang and A. Zhamu, published in the Journal of Materials Science, August 2008, Volume 43.15, pages 5092-5101.

The adsorbents are usually hydrophobic. The adsorbents can then be treated to reduce their hydrophobicity, enabling them to be more easily dispersed in the polymer matrix.

To do this, it is possible for example to soak them in a solution of ethanol and then to allow the ethanol to evaporate.

The adsorbents are not carbon black.

The adsorbents of a composition according to the invention may have a specific surface area greater than 300 m²/g, preferably greater than 500 m²/g, more preferably greater than 700 m²/g, even more preferably greater than 1000 m²/g.

The adsorbents and in particular the activated carbon particles used in the invention have for example an average size of less than 1 millimeter. The activated carbon particles used in the invention are for example powdered activated carbon (R 1, PAC), or bead activated carbon (BAC).

In some embodiments, the adsorbents and in particular the activated carbon particles used in the invention may have an average size of less than 200 microns for example.

The adsorbents and in particular the activated carbon particles may have a carbon content of less than 90% by weight.

The adsorbents are preferably homogeneously dispersed in the polymer matrix.

The term “homogeneously dispersed” is understood to mean that the adsorbents do not form aggregates, in particular aggregates having a size greater than 1 mm.

Electrical communication in living tissue, and in particular the human body, is mainly carried out by flows of charged ions such as Cl−, K+, and/or Na+. These movements of charged ions generate changes in the electric or magnetic potential, which can be measured outside the body and provide information about its function.

To use an electrode, it is therefore possible to capture the ions present on the surface of living tissue 9, in particular the skin of a person 9, and to convert those ions into an electrical flow in an electrical circuit for signal processing.

To do so, the adsorbents provide a large adsorption specific surface area able to adsorb a large amount of ions present on the surface of the living tissue in contact with the polymer composition.

Furthermore, carbon nanotubes are used to render the polymer composition conductive.

The adsorption of ions on the adsorbents therefore causes a change in potential in the entire polymer composition, a change in potential that can then be transmitted directly to an electrical circuit simply connected to the potential of the polymer composition.

In this regard, the carbon nanotubes render the polymer composition conductive with a relatively low percentage of material.

In particular, the percentage by weight of carbon nanotubes in the polymer matrix corresponding to an electrical percolation threshold of carbon nanotubes in the polymer matrix, is in particular less than a percentage of adsorbents by weight in the polymer matrix corresponding to an electrical percolation threshold of adsorbents in the polymer matrix.

To provide clarification by giving a purely non-limiting example, the electrical percolation threshold is usually reached at about 5% carbon nanotubes by weight in a polymer matrix, while it takes more than 30% active carbon by weight to form a percolation network.

The advantage of incorporating a small amount of carbon nanotubes and adsorbents is maintaining the mechanical properties of the polymer matrix.

With a large amount of adsorbents, the polymer composition becomes very rigid, which reduces the comfort and effectiveness of the electrode.

In fact, to maximize adsorption of ions in the polymer composition and comfort during use, it is of interest that the polymer composition has a low hardness, so that it can easily follow the curves of the living tissue with which it is in contact, particularly a skin surface.

For example, the polymer composition comprises from 0.5 to 10 percent carbon nanotubes by weight, preferably from 0.5 to 6 percent carbon nanotubes by weight, based on the total weight of the polymer composition.

Moreover, the polymer composition may comprise from 0.5 to 30 percent adsorbents by weight, preferably from 0.5 to 15 percent adsorbents by weight, based on the total weight of the polymer composition.

In general, a percentage by weight of adsorbents in the polymer composition can be less than a percentage by weight of adsorbents in the polymer matrix corresponding to an electrical percolation threshold for adsorbents in the polymer matrix.

The polymer composition can then have a hardness within a range from 10 Shore A to 80 Shore A. The hardness of the polymer composition may be less than 65 Shore A.

Preparation:

The polymer composition defined above can be prepared using a twin-screw extruder with screws rotating in the same direction, for example a 25 mm diameter twin-screw extruder of the brand Berstorff GmbH.

The elements of the polymer composition can be fed in several ways. Either the set of elements is fed into the feed throat of the extruder and passes through said extruder, or the carbon nanotubes and/or the adsorbents are fed by a side feeder and the elements of the polymer matrix are fed by the throat. The polymer composition can then be vacuum degassed.

Other techniques for mixing in the molten state and for composition preparation such as single-screw extruders and Banbury mixers may also be used.

In another embodiment of the invention, the polymer matrix may be a crosslinkable polymer matrix and the carbon nanotubes and/or adsorbents may be mixed with the crosslinkable matrix in liquid phase before the matrix is crosslinked in the amorphous phase.

In some modes of preparation, functionalization of the adsorbents and/or carbon nanotubes may be achieved by coupling agents and/or capping agents enabling their homogeneous dispersion in the polymer matrix.

Crosslinking of the polymer matrix may then be performed by UV and/or by heating. A catalyst may be added to initiate or promote the crosslinking, for example a free radical photoinitiator.

Measurement Methods:

The specific surface area of activated carbon particles can be measured by the subtracting pore effect method described in the article “Origin of superhigh Surface Area and microcrystalline graphitic structures of activated carbons” by Kaneko, K., C. Ishii and M. Ruike published in Carbon, vol 30.7 (1992): pages 1075-1088.

The specific surface area of graphene nanoplatelets can be measured by the Brunauer, Emmett and Teller (BET) nitrogen adsorption method, for example as indicated in the article “Processing of nanographene platelets (NGPs) and NGP nanocomposites: a review” by B. Z. Jang and A. Zhamu, published in the Journal of Materials Science, August 2008, Volume 43.15, pages 5092-5101.

The hardness of the polymer composition can be measured using a Shore durometer.

Electrode:

The invention also relates to an electrode 6 for the non-invasive measurement of biological electrical signals, in particular without conductive gel. The electrode 6 comprises a polymer composition 1 as described above, arranged to be suitable for forming an interface with living tissue 9.

Thus, the polymer composition 1 may be shaped in the electrode 6 to form a first layer 1 extending between a first face 1a, able to come into contact with living tissue 9, and a second face 1b opposite to the first face in a thickness direction Z.

In one embodiment of the invention, the polymer composition 1 may be in direct contact with electrical conductors 5, in particular electrical wires connected to a unit 8 for processing the signals measured by the electrode. The electrical conductors 5 may be incorporated into the polymer composition or may be placed in contact with one face of the polymer composition, for example the second face 1b.

The electrode 6 and the unit 8 for processing the signals measured by the electrode can thus form an electrical circuit 7 for the non-invasive measurement of biological electrical signals.

In an alternative embodiment illustrated in FIG. 2, the electrode 6 may further comprise a second layer 10 of a conductive polymer 10. The second layer may be in contact with the polymer composition 1, for example being placed on the second face 1b of the polymer composition 1.

In this alternative embodiment, the polymer composition 1 may be in contact with the electrical conductors 5 by means of the second layer 10 of conductive polymer.

Thus, the electrical conductors 5 may be incorporated into the second layer 10 or may be placed in contact with a face 10b of the second layer 10.

In particular, the second layer 10 may be shaped in the electrode 6 to extend between a first face 10a in contact with the second face 1b of the first layer 1, and a second face 10b opposite to the first face 10a in the thickness direction Z.

The electrical conductors 5 can then for example be placed in contact with the second face 10b of the second layer 10.

In one embodiment, the thickness of the polymer composition 1 measured in the thickness direction Z may then be less than the thickness of the conductive polymer 10 measured in the thickness direction Z. The thickness of the polymer composition 1 may in particular be at least two times smaller than the thickness of the conductive polymer 10.

The presence of a second layer of conductive polymer allows greater freedom in the mechanical properties for the electrode.

For example, the conductive polymer may have a hardness greater than the hardness of the polymer composition.

In an exemplary embodiment of the invention, the conductive polymer may be a thermoplastic polyurethane (TPU). The conductive polymer may in particular be doped with carbon nanotubes in a similar manner to what was described above for the polymer composition, but without requiring the presence of adsorbents.

The polymer composition and/or the conducting polymer may be shaped by injection molding, fiber spinning, extrusion, or compression molding, and by combinations of these techniques.

In the case of injection molded electrodes, an overmolding technique may be used in which electrical conductors are pre-positioned in a mold into which the polymer composition and/or the conductive polymer is injected. Alternatively, an electrode may be molded, then the electrical conductors are soldered to the electrode, in particular to the polymer composition and/or the conductive polymer. Similarly, one among the polymer composition and the conductive polymer may be molded and the other among the polymer composition and the conductive polymer may be overmolded.

For extruded electrodes, the polymer composition and/or the conducting polymer may be coextruded together and/or with the electrical conductors, then cut into electrode shapes. Alternatively, one or more among the polymer composition and the conducting polymer may be extruded into a film, sheet, strip, or foam, and then the electrical conductors may be welded or introduced into the polymer composition and/or the conductive polymer. The electrical conductors may also be produced by metal deposition techniques on the polymer composition and/or conductive polymer or on the formed electrode.

Alternatively, the polymer composition and/or the conducting polymer may be formed into fibers and woven into a fabric or clothing.

Electrical Circuit:

The invention further relates to an electrical circuit 7 incorporating an electrode 6 as described above, comprising at least one electrical conductor 5 electrically connected to the polymer composition 1 or to the conductive polymer 10. The electrical circuit 7 further comprises a unit 8 for processing the signals measured by the electrode. The processing unit 8 is electrically connected to the electrical conductor 5 of the electrode 6 as described above.

The processing unit 8 may in particular comprise an instrumentation amplifier and/or filter modules such as band-pass or low-pass filters for example.

Measurement Device:

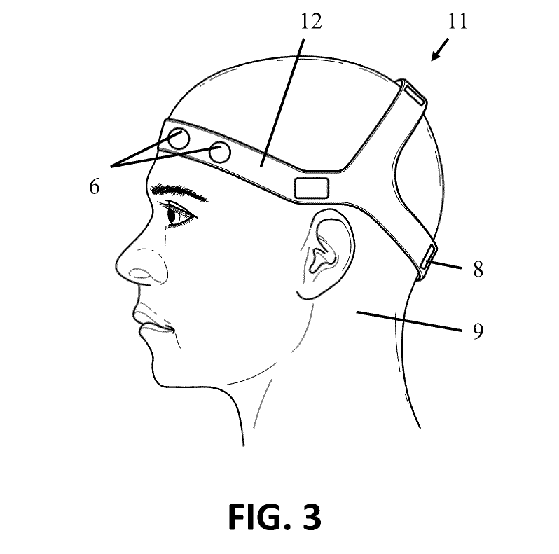

Lastly, the invention relates to a device 11 for measuring brain waves, suitable for wearing by a person 9, schematically illustrated in FIG. 3. The device 1 comprises a support member 12 adapted to at least partially surround the head of a person 9 so as to be held thereon.

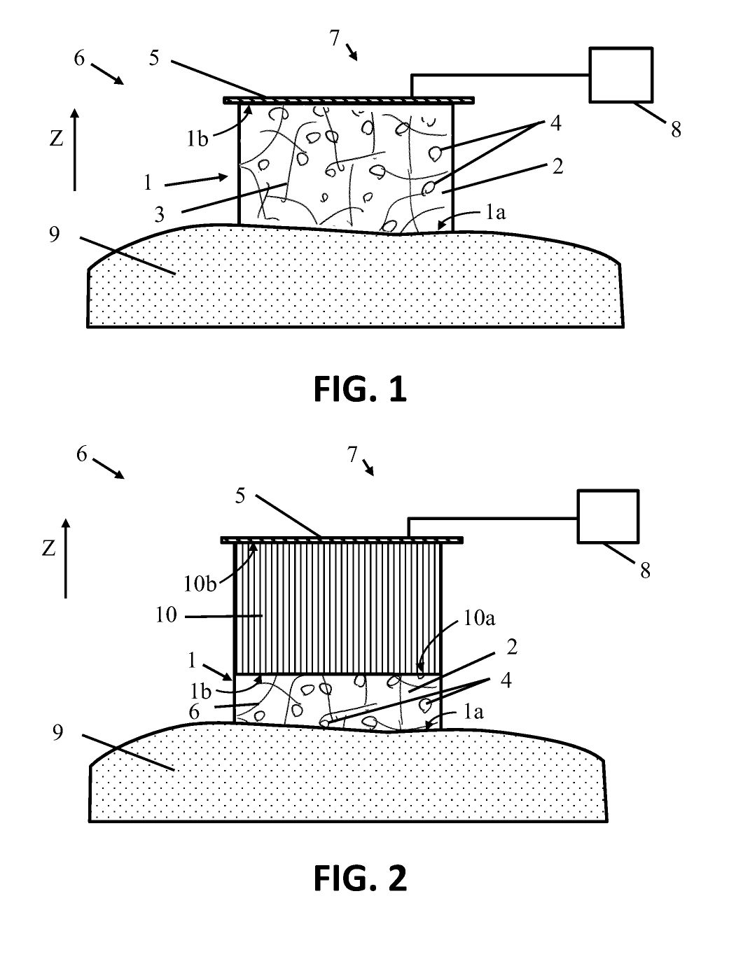

The support member 12 is adapted to at least partially surround the head of the person 9 so as to be held thereon. In one embodiment of the invention illustrated in FIG. 1, the support member 12 is adapted in particular to surround at least a portion of the circumference of the head of the person 9, in particular to surround at least half the circumference of the head of the person 9 or even to entirely surround a diameter of the head of the person 9.

In the embodiment illustrated in FIG. 3, the support member 12 may comprise several interconnected arms which surround different portions of the head of the person 9 so as to ensure stable retention and precise positioning of the device 11 on the person 9.

At least one electrode 6 as described above is mounted on the support member 12 such that the polymer composition is able to come into contact with the skin of the person 9.

The electrode 6 is for example mounted on an inner face of the support member 12 which is able to come into contact with the person's skin.

In one embodiment, the device 11 comprises an electrical circuit 7 as described above. In this embodiment, the processing unit 8 may in particular be mounted on the support member 12.

1-13. canceled

14. Polymer composition comprising a polymer matrix in which are dispersed carbon nanotubes and adsorbents selected from activated carbon particles and graphene nanoplatelets.

15. Polymer composition according to claim 14, comprising from 0.5 to 10 percent carbon nanotubes by weight, based on the total weight of the polymer composition.

16. Polymer composition according to claim 14, comprising from 0.5 to 30 percent adsorbents by weight, based on the total weight of the polymer composition.

17. Polymer composition according to claim 14, wherein the adsorbents have a specific surface area greater than 300 m²/g.

18. Polymer composition according to claim 14, wherein the polymer composition has a hardness within a range of 10 Shore A to 80 Shore A.

19. Electrode for the non-invasive measurement of biological electrical signals, the electrode comprising a polymer composition according to claim 14 that is able to come into contact with living tissue.

20. Electrode according to claim 19, wherein the polymer composition forms a first layer extending between a first face, able to come into contact with living tissue, and a second face opposite to the first face in a thickness direction, and wherein the electrode further comprises a second layer of a conductive polymer arranged on the second face of the polymer composition.

21. Electrode according to claim 20, wherein a thickness of the first layer, measured in the thickness direction, is smaller than a thickness of the second layer.

22. Electrode according to claim 20, wherein the conductive polymer has a hardness greater than a hardness of the polymer composition.

23. Electrical circuit for the non-invasive measurement of biological electrical signals, comprising
an electrode according to claim 19, comprising one electrical conductor in contact with the polymer composition or the conductive polymer,
a unit for processing the signals measured by the electrode, connected to the electrical conductor of the electrode.

24. Device for measuring brain waves, suitable for wearing by a person, the device comprising a support member, adapted to at least partially surround the head of the person so as to be held thereon, on which is mounted one electrode according to claim 19 such that the polymer composition of the electrode is able to come into contact with the skin of said person.

25. Device for measuring brain waves, suitable for wearing by a person, the device comprising a support member, adapted to at least partially surround the head of the person so as to be held thereon, on which is mounted one electrode for the non-invasive measurement of biological electrical signals, the electrode comprising a polymer composition comprising a polymer matrix in which are dispersed carbon nanotubes and adsorbents selected from activated carbon particles and graphene nanoplatelets, the polymer composition being suitable for contact with living tissue, such that the polymer composition of the electrode is able to come into contact with the skin of said person, the device comprising an electrical circuit according to claim 23 wherein the electrode and the processing unit are mounted on the support member.

26. A method for increasing the ionic adsorption of a polymer composition comprising a polymer matrix in which carbon nanotubes are dispersed, comprising providing and applying adsorbents selected from activated carbon particles and graphene nanoplatelets, wherein said adsorbents being dispersed in the polymer matrix.

27. The polymer composition of claim 15, comprising 2 to 6 percent carbon nanotubes by weight, based on the total weight of the polymer composition.

28. The polymer composition of claim 16, comprising from 2 to 15 percent adsorbents by weight, based on the total weight of the polymer composition.

29. The polymer composition of claim 17, wherein the adsorbents have a specific surface area greater than 500 m²/g.

30. The polymer composition of claim 17, wherein the adsorbents have a specific surface area greater than 700 m²/g.

31. The polymer composition of claim 17, wherein the adsorbents have a specific surface area greater than 1000 m²/g.

32. The electrode of claim 21, wherein a thickness of the first layer, measured in the thickness direction, is two times smaller than a thickness of the second layer.

33. Device for measuring brain waves, suitable for wearing by a person, the device comprising a support member, adapted to at least partially surround the head of the person so as to be held thereon, on which is mounted one electrode according to claim 20 such that the polymer composition of the electrode is able to come into contact with the skin of said person.
